(19) United States
(12) Patent Application Publication
(10) Pub. No.: US 2022/0061473 A1
(43) Pub. Date: Mar. 3, 2022

(54) HAND SANITIZING SYSTEM, BRACELET APPLICATOR APPARATUS, DISPENSING SYSTEM, REFILL SYSTEM, AND METHOD OF USE

(71) Applicant: Fernando Newcomb, Hong Kong (HK)

(72) Inventor: Fernando Newcomb, Hong Kong (HK)

(21) Appl. No.: 17/460,408

(22) Filed: Aug. 30, 2021

Related U.S. Application Data
(60) Provisional application No. 63/072,267, filed on Aug. 31, 2020.

(51) Int. Cl. A44C 5/00; A61L 2/00; A44C 5/20

(52) U.S. Cl. CPC A44C 5/003; A61L 2/0088; A61L 2202/16; A61L 2202/15; A44C 5/2071

(57) ABSTRACT

A hand sanitizing system, bracelet applicator apparatus, dispensing system, refill system, and method of use are presented. The bracelet disinfectant system is configured to be worn on a user's wrist. The system is located close to the palm of the user. The bracelet disinfectant system ejects a predetermined amount of sanitizing solution onto the palm of the user. The ejection of the solution can be completed without removing the bracelet from the wrist of the user. The system provides a disinfectant bracelet and refillable system that can be caused to eject a sanitizing solution from the system to the palm of a user. The system provides a user quick and easy ejection of a predetermined amount of solution onto a single palm and still have both hands free to rub the solution into their hands and/or onto another surface—so as to make the surface disinfected.

### CROSS-REFERENCE TO RELATED APPLICATIONS

The present application claims priority to the U.S. Provisional Patent Application No. 63/072,267 which was filed on Aug. 31, 2020, which is hereby incorporated by reference herein in its entirety, including any figures, tables, or drawings.

### FIELD OF THE DISCLOSURE

The present disclosure relates generally to a hand sanitizing system, bracelet applicator apparatus, dispensing system, refill system, and method of use. More specifically, and without limitation, the present disclosure is a device with multi-function capability as both a bracelet apparatus, sanitizer system, and an all inclusive dispenser and refill system, and method of use which provides for convenient, on demand hand sanitization. The present disclosure is specifically designed to make hand sanitizing easy and convenient, whatever the circumstances. However, the present disclosure is not limited to these novel and inventive improvements, and it may further be adapted for a variety of purposes.

### COPYRIGHT NOTICE

At least a portion of the disclosure of this patent document contains material that is subject to copyright protection. The copyright owner has no objection to the facsimile reproduction by anyone of the patent document or the patent disclosure, as it appears in the Patent and Trademark Office patent files and/or records, but otherwise reserves all copyright rights whatsoever. The following notice applies to the software and data as described below and in the drawings that form a part of this document. Copyright Sentinel Systems Limited. All rights reserved.

### BACKGROUND OF THE DISCLOSURE

Hand sanitizing is well known in the art. Typically, hand sanitizing is completed by washing hands. In fact, the center for disease control recommends washing hands with soap and water whenever possible to reduce amounts of germs and chemicals on hands. Soap and water are not always readily available. This may exist when a person is in a location without access to running water and soap, such as in a car, in a grocery store aisle, or another retail location and the like. For these circumstances where hand washing is not readily available or convenient, hand sanitizer is a popular alternative.

Hand sanitizer is a formula and/or mixture commonly comprising at least 60% alcohol and/or other formulas and mixtures that kill certain kinds of germs such as coronavirus, other viruses and the like (alcohol free versions of hand sanitizer exist, but are less common). Hand sanitizers are commonly found in liquid, gel, or foam form and are effective once dispensed and rubbed over the surface of hands of a user, killing germs and the like on the surface of human hands or along other locations of application.

Existing hand sanitizer methods include a pump bottle and the like. Pump bottle hand sanitizers are common for countertop and/or table tops and the like. Pump bottles provide a pump that when pushed sprays a set amount of hand sanitizer into the waiting hand of a user. This is one method of hand sanitizer dispersion and is common in public places and has become popular in times of disease spread such as a pandemic. Pump bottles, however, require every user to press the bottle, potentially encouraging the spread of disease. Furthermore, pump bottles are unlikely to be carried with a user as they are bulky and can easily leak within a purse or bag.

Another common hand sanitizer dispenser is a squeeze bottle. Squeeze bottles apply an indeterminable amount of hand sanitizer to a waiting set of human hands when the bottle is opened—removing a lid—and then the bottle is squeezed to dispense hand sanitizer. The hand sanitizer bottle lid must then be closed and the bottle put away before the sanitizer can be rubbed in. In this way, a user can apply the product to their hands, rub the hands together and let the product dry over all surfaces of the hands for successful application. Carrying the bottle and dispensing and fumbling the bottle remain problematic.

The advent of the covid-19 pandemic has further strained the demands on the need for hand sanitizing in order to reduce the transmission of infectious diseases such as the coronavirus disease 2019 (covid-19) among other viruses, microbes, disease, and the like. Covid-19 has proven to be one of many viruses that can live on surfaces for long durations of time. In such a circumstance, it becomes important for individuals to sanitize their hands regularly, such as after door handles are used, elevator buttons are pressed, credit cards are handled, packages are handled, and the like. Thus, it has become more important than ever to have regular and easy and convenient access to and delivery of an optimal dose of hand sanitizing solution to cleanse the hands and/or rub the solution on parts of a user.

Complicating matters further, due to shortages of hand sanitizer and the like, many entities have resorted to using hand sanitizers which come from unreliable sources and/or are produced by unreliable entities. In some circumstances, harmful chemicals and/or toxins have been found in many hand sanitizers available on the market. This problem has become so prevalent that the federal drug administration (FDA) has created a list of sanitizers it recommends against using. Controlling the use of what type of hand sanitizer and/or the source of that hand sanitizer is not always easy as hand sanitizers available in public, such as at stores, and the like, are not always labeled. Therefore, this is another reason to have easy access to a reliable hand sanitizer on a regular basis.

In addition to overcoming these challenges and hurdles for proper implementation of proper sanitization to fight the spread of infectious disease and to overcome hurdles of protecting a others from the spread of infectious diseases, individuals, companies and the like must also consider hygiene and the regular use of hand sanitizers in the workplace as well as individuals considered regular use of hand sanitizers in everyday life.

In order to prevent irritation or inconvenience, a reusable, readily available hand sanitizing device is needed so humans can have access to cleanliness on a regular basis. For this reason, and oftentimes to avoid liability associated with health conditions caused by infectious diseases living on the surface, disposable or single-use sanitizing wipes are sometimes used. While these are more convenient, they often lead to undesirable waste and require a trash can for disposal. Disposable or single-use wipes are intended for a single use and are not washed for reuse. Additionally, for single use applications, correct implementation becomes even more of a challenge for ease and regular use. For this reason, and many others, single use wipes have not experienced widespread and regular usage as is needed to prevent the spread of infectious diseases and control hygiene.

Hygiene is just one of many issues that exist in the implementation and use of a hand sanitizing device. Hand sanitizer is often misused, hard to apply, hard to carry, hard to store, and the like. It is estimated that when available, most of the time an improper dosage is applied, is misused or is stored in a way that damages the product itself or compromises the integrity of the product.

Thus, there is a need in the art for a more convenient hand sanitizing system that can be used regularly and easily for individuals navigating surfaces and for companies to implement with employees in the workplace. This will prevent a need for hand washing on a constant basis, add to productivity, and prevent the spread of infectious diseases, while improving the health and well being of the user and others around or interacting with the user.

It is apparent that a need exists for a novel solution to provide a more hygenic, easy to use, easy to implement, and create a more readily available, more cost effective solution to hand sanitization and/or use of a hand sanitizer. The present disclosure is directed toward providing such a solution.

### SUMMARY OF THE DISCLOSURE

The purpose of this disclosure is to provide the state of the art with a hand sanitizing system, bracelet applicator apparatus, dispensing system, refill system, and method of use. More specifically, and without limitation, the present disclosure is a device with multi-function capability as both a bracelet apparatus, sanitizer system, and an all inclusive dispenser and refill system, and method of use which provides for convenient, on demand, convenient hand sanitization. The present disclosure is specifically designed to make hand sanitizing easy and convenient, whatever the circumstances. However, the present disclosure is not limited to these novel and inventive improvements, and it may further be adapted for a variety of purposes.

More specifically, and without limitation, the present disclosure is a device with multi-function capability as both a bracelet and sanitizing system, and an all inclusive dispenser, and method of use which provides for dispensing a dosage of sanitizer when needed. The present disclosure is specifically designed to make the correct dosage available on demand and on a regular and convenient basis for proper safety protection and maximizing the effectiveness of the control of the spread of infectious disease.

Furthermore, the present disclosure offers convenient and safe storage for a sanitizing solution. In this way, a user can have convenient access to the sanitizing solution wherever that user may be. The storage solution this disclosure presents combines an effective and convenient storage means that is also easy to access. In one example, the bracelet presented herein is lightweight and comfortable, and oftentimes unnoticed, by a user, being located on the wrist of a user.

In combination with convenience, the present disclosure offers a hygienic solution of a bracelet and dispensing system which prevents infection and other hygienic problems caused by the mishandling and/or the lack of convenience associated with most carry bottles, pump bottles, sanitizer stations, hand washing, and the like. Single use and/or disposable sanitizing systems increase the amount of waste and themselves present a convenience issue.

For this reason, and in addition to the above, the disclosure herein presents a convenient, sanitizing dispenser system.

The present disclosure provides a wearable which includes a refillable sanitizer supply that delivers on demand dosages of sanitizer. In this way, a wearer can access sanitizer conveniently any time, anywhere and on a regular basis.

Through a solution that incorporates convenience, hygienic advantages along with safety advantages, as well as proper deployment, the disclosure herein presents a much improved, novel, and needed sanitizer dispensing system and method of use.

Said another way, the disclosure consists of three unique elements intended to be claimed separately as well as a method of use and/or application. First, the present disclosure presents a convenient wearable that is comfortable and that improves upon the convenience and dispensary advantages of the state of the art. Second, the present disclosure presents a convenient applicator that improves upon the hygienic state of the art—through proper dosage delivery and the like. Third, the present disclosure presents a convenient refillable dispensing system that improves upon the effectiveness and/or greatly increases the proper and safe use of the sanitizing solution and/or sanitizing formula.

Taken individually, all the above provide for effective improvements upon the state of the art. Additionally, taken in combination, this disclosure provides for a convenient, easy to use, hygienic, effective wearable, applicator and method of use that greatly improves upon the state of the art and/or provides a novel device and methodology of using a access to and dispersion of a sanitizing solution.

Thus, it is a primary object of the disclosure to provide a hand sanitizing system, bracelet applicator apparatus, dispensing system, refill system, and method of use that improve upon the state of the art.

Another object of the disclosure is to provide a hand sanitizing system, bracelet applicator apparatus, dispensing system, refill system, and method of use that provides a comfortable wearable.

Yet another object of the disclosure is to provide a hand sanitizing system, bracelet applicator apparatus, dispensing system, refill system, and method of use that provides a lightweight wearable.

Another object of the disclosure is to provide a hand sanitizing system, bracelet applicator apparatus, dispensing system, refill system, and method of use that can be accessed anywhere.

Yet another object of the disclosure is to provide a hand sanitizing system, bracelet applicator apparatus, dispensing system, refill system, and method of use that can be dispensed any time.

Another object of the disclosure is to provide a hand sanitizing system, bracelet applicator apparatus, dispensing system, refill system, and method of use that can be dispensed in seconds.

Yet another object of the disclosure is to provide a hand sanitizing system, bracelet applicator apparatus, dispensing system, refill system, and method of use that provides for convenient hand sanitizing.

Another object of the disclosure is to provide a hand sanitizing system, bracelet applicator apparatus, dispensing system, refill system, and method of use that emits an equal dosage each use.

Yet another object of the disclosure is to provide a hand sanitizing system, bracelet applicator apparatus, dispensing system, refill system, and method of use that can be pumped.

Another object of the disclosure is to provide a hand sanitizing system, bracelet applicator apparatus, dispensing system, refill system, and method of use that provides a storage tank.

Yet another object of the disclosure is to provide a hand sanitizing system, bracelet applicator apparatus, dispensing system, refill system, and method of use that provides a refillable capsule.

Another object of the disclosure is to provide a hand sanitizing system, bracelet applicator apparatus, dispensing system, refill system, and method of use that provides a sprayer.

Yet another object of the disclosure is to provide a hand sanitizing system, bracelet applicator apparatus, dispensing system, refill system, and method of use that provides an emitter.

Another object of the disclosure is to provide a hand sanitizing system, bracelet applicator apparatus, dispensing system, refill system, and method of use that utilizes replaceable cartridges.

Yet another object of the disclosure is to provide a hand sanitizing system, bracelet applicator apparatus, dispensing system, refill system, and method of use that is fashionable.

Another object of the disclosure is to provide a hand sanitizing system, bracelet applicator apparatus, dispensing system, refill system, and method of use that is easy to wear.

Yet another object of the disclosure is to provide a hand sanitizing system, bracelet applicator apparatus, dispensing system, refill system, and method of use that is comfortable to wear on a wrist.

Another object of the disclosure is to provide a hand sanitizing system, bracelet applicator apparatus, dispensing system, refill system, and method of use that can be used with interchangeable bands.

Yet another object of the disclosure is to provide a hand sanitizing system, bracelet applicator apparatus, dispensing system, refill system, and method of use that is adjustable for various sizes.

Another object of the disclosure is to provide a hand sanitizing system, bracelet applicator apparatus, dispensing system, refill system, and method of use that comes with a bottle changing feature.

Yet another object of the disclosure is to provide a hand sanitizing system, bracelet applicator apparatus, dispensing system, refill system, and method of use that has an easy to open lid.

Another object of the disclosure is to provide a hand sanitizing system, bracelet applicator apparatus, dispensing system, refill system, and method of use that has an easy to close and seal lid.

Yet another object of the disclosure is to provide a hand sanitizing system, bracelet applicator apparatus, dispensing system, refill system, and method of use that locks in place.

Another object of the disclosure is to provide a hand sanitizing system, bracelet applicator apparatus, dispensing system, refill system, and method of use that is available in various shapes and sizes.

Yet another object of the disclosure is to provide a hand sanitizing system, bracelet applicator apparatus, dispensing system, refill system, and method of use that is sleek.

Another object of the disclosure is to provide a hand sanitizing system, bracelet applicator apparatus, dispensing system, refill system, and method of use that is easy to operate.

Yet another object of the disclosure is to provide a hand sanitizing system, bracelet applicator apparatus, dispensing system, refill system, and method of use that quickly operates by the touch of a finger.

Another object of the disclosure is to provide a hand sanitizing system, bracelet applicator apparatus, dispensing system, refill system, and method of use that is easy to use.

Yet another object of the disclosure is to provide a hand sanitizing system, bracelet applicator apparatus, dispensing system, refill system, and method of use that is safe.

Another object of the disclosure is to provide a hand sanitizing system, bracelet applicator apparatus, dispensing system, refill system, and method of use that is comfortable.

Yet another object of the disclosure is to provide a hand sanitizing system, bracelet applicator apparatus, dispensing system, refill system, and method of use that is an all in one integrated system.

Another object of the disclosure is to provide a hand sanitizing system, bracelet applicator apparatus, dispensing system, refill system, and method of use that are hygienic.

Yet another object of the disclosure is to provide a hand sanitizing system, bracelet applicator apparatus, dispensing system, refill system, and method of use that are convenient.

Another object of the disclosure is to provide a hand sanitizing system, bracelet applicator apparatus, dispensing system, refill system, and method of use that improves upon the convenience of the state of the art.

Yet another object of the disclosure is to provide a hand sanitizing system, bracelet applicator apparatus, dispensing system, refill system, and method of use that are easy to access.

Another object of the disclosure is to provide a hand sanitizing system, bracelet applicator apparatus, dispensing system, refill system, and method of use that can be refilled rapidly.

These and other objects, features, or advantages of the present disclosure will become apparent from the specification and claims.

### BRIEF DESCRIPTION OF THE DRAWINGS

FIG. 1 is a top, perspective view showing the hand sanitizing system, bracelet applicator apparatus, dispensing system, and refill system; the view showing a band; the view showing a sprayer; the view showing an actuator.

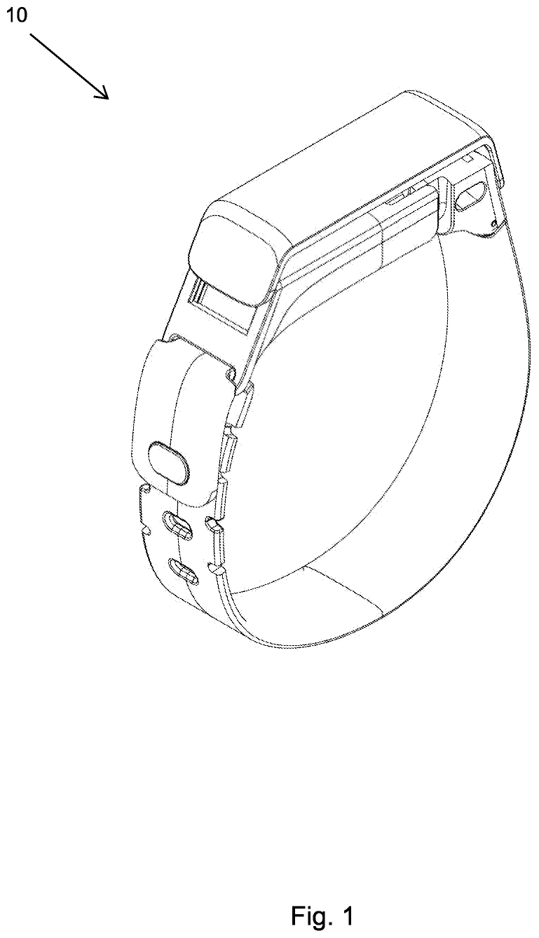

FIG. 2 is a top, perspective view showing the hand sanitizing system, bracelet applicator apparatus, dispensing system, and refill system; the view showing a band; the view showing a sprayer; the view showing an actuator.

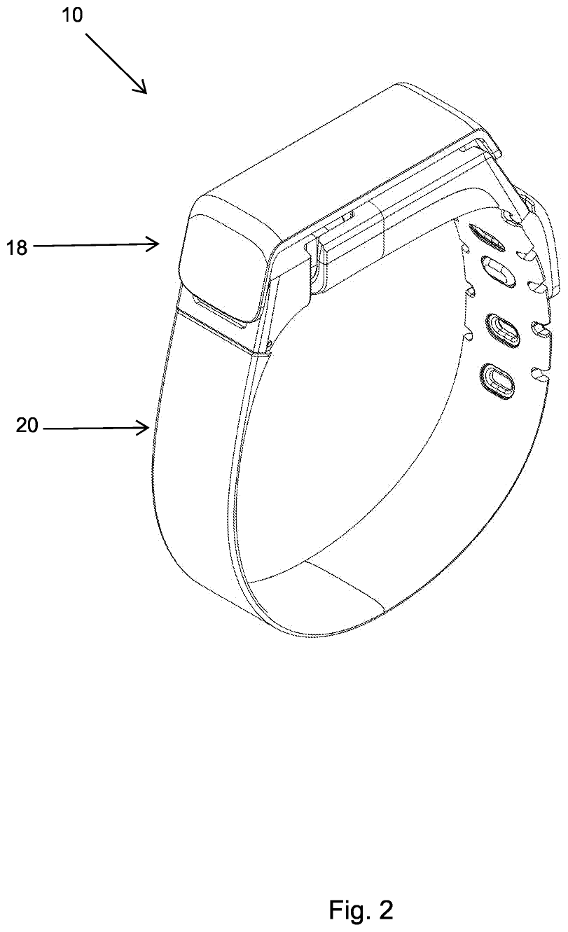

FIG. 3 is a top view showing the hand sanitizing system, bracelet applicator apparatus, dispensing system, and refill system; the view showing a band; the view showing a sprayer; the view showing an actuator.

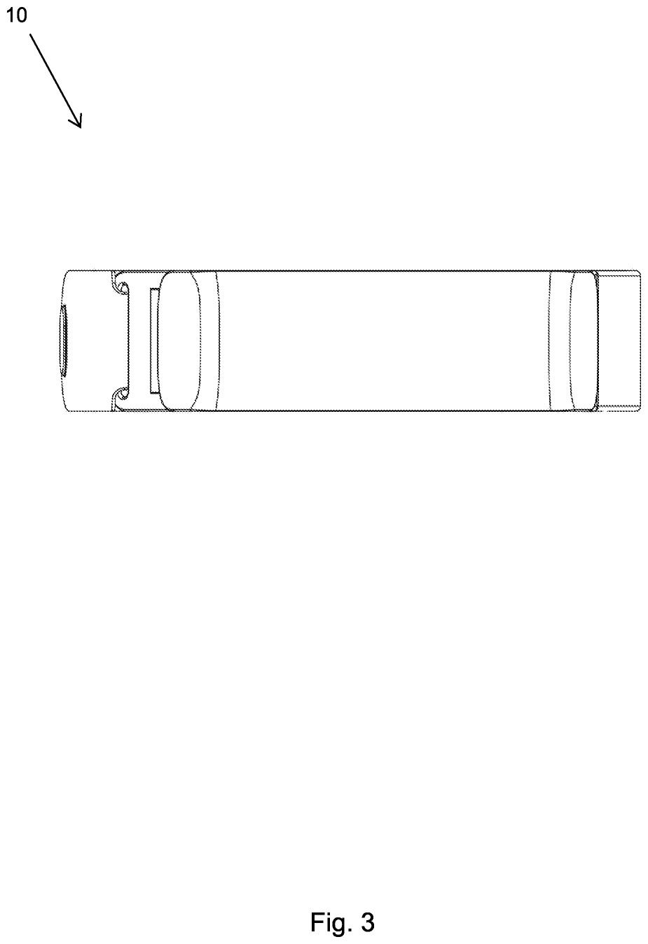

FIG. 4 is a bottom view showing the hand sanitizing system, bracelet applicator apparatus, dispensing system, and refill system; the view showing a band; the view showing a sprayer; the view showing an actuator.

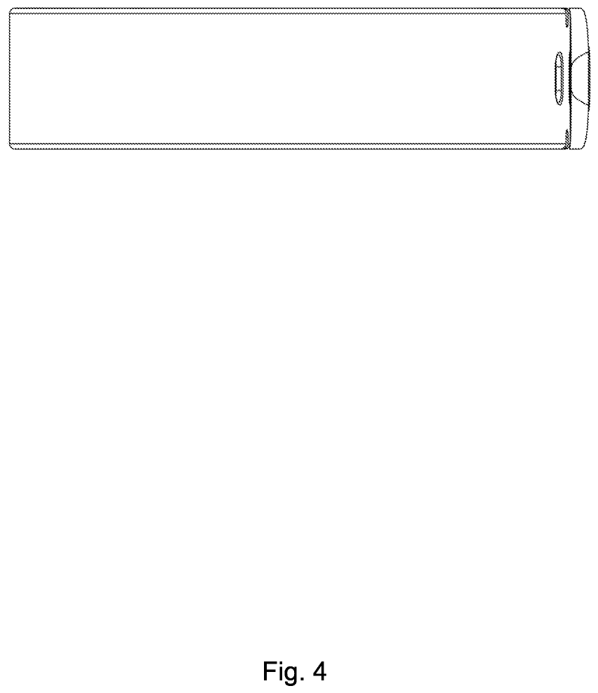

FIG. 5 is a front view showing the hand sanitizing system, bracelet applicator apparatus, dispensing system, and refill system; the view showing a band; the view showing a sprayer; the view showing an actuator.

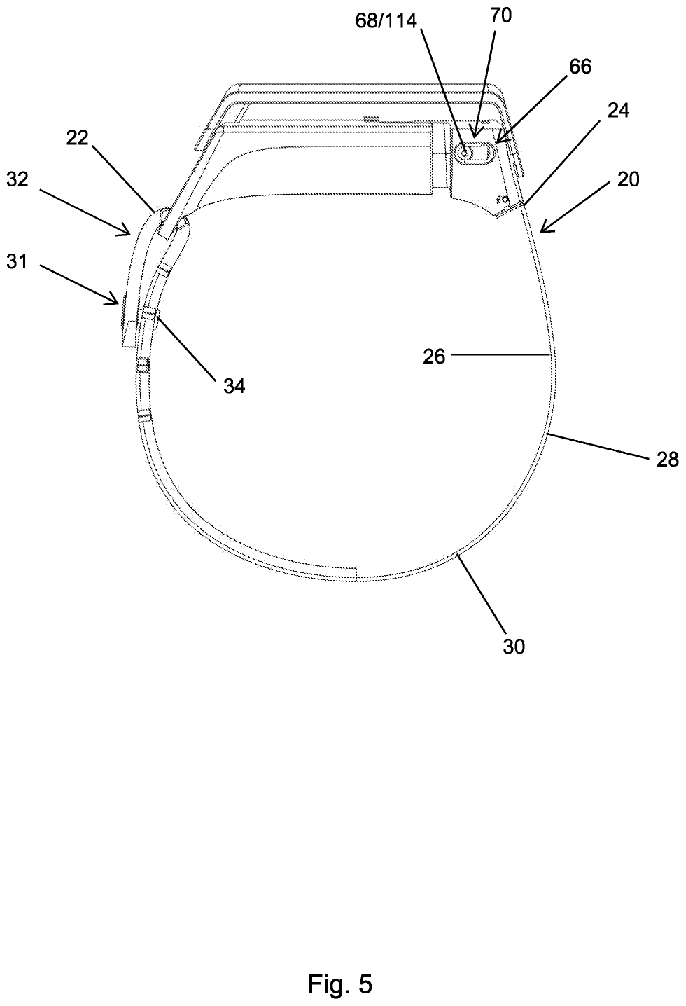

FIG. 6 is a rear view showing the hand sanitizing system, bracelet applicator apparatus, dispensing system, and refill system; the view showing a band; the view showing a sprayer; the view showing an actuator.

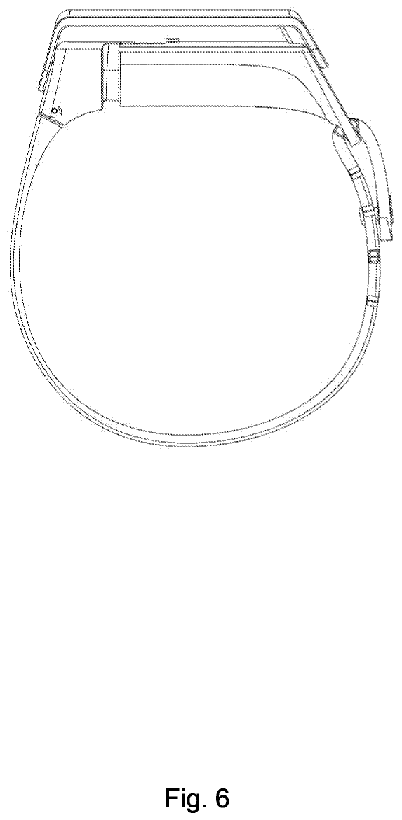

FIG. 7 is a side elevation view showing the hand sanitizing system, bracelet applicator apparatus, dispensing system, and refill system; the view showing a band; the view showing a sprayer; the view showing an actuator.

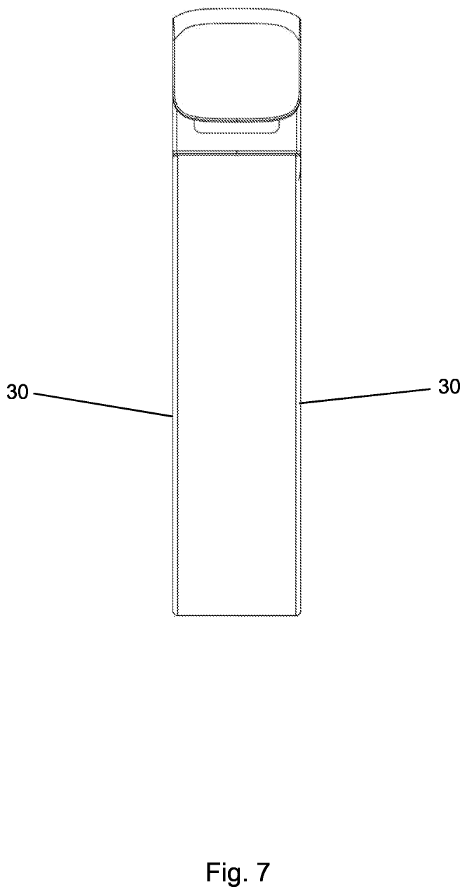

FIG. 8 is a side elevation view showing the hand sanitizing system, bracelet applicator apparatus, dispensing system, and refill system; the view showing a band; the view showing a sprayer; the view showing an actuator.

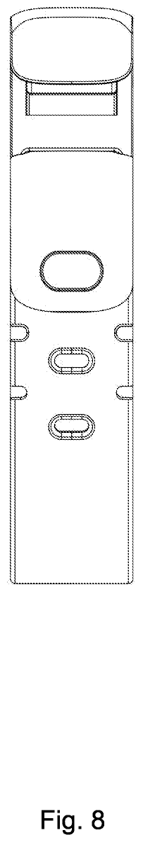

FIG. 9 is a bottom, perspective view showing the hand sanitizing system, bracelet applicator apparatus, dispensing system, and refill system; the view showing a band; the view showing a sprayer; the view showing an actuator.

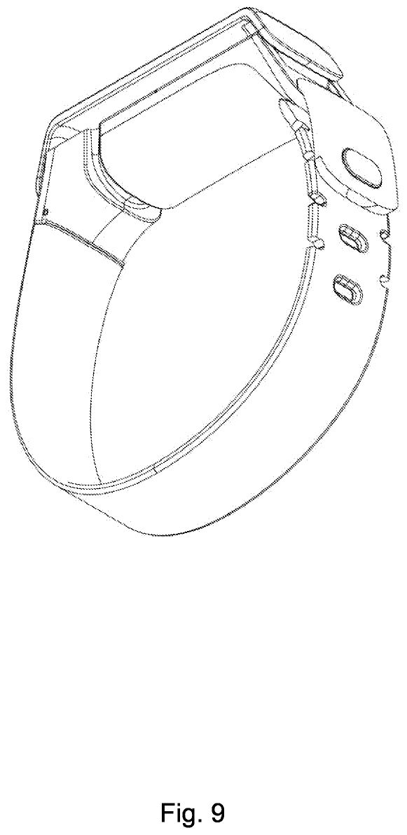

FIG. 10 is a top, perspective, exploded view showing the hand sanitizing system, bracelet applicator apparatus, dispensing system, and refill system; the view showing a band; the view showing a sprayer; the view showing an actuator; the view showing a cartridge.

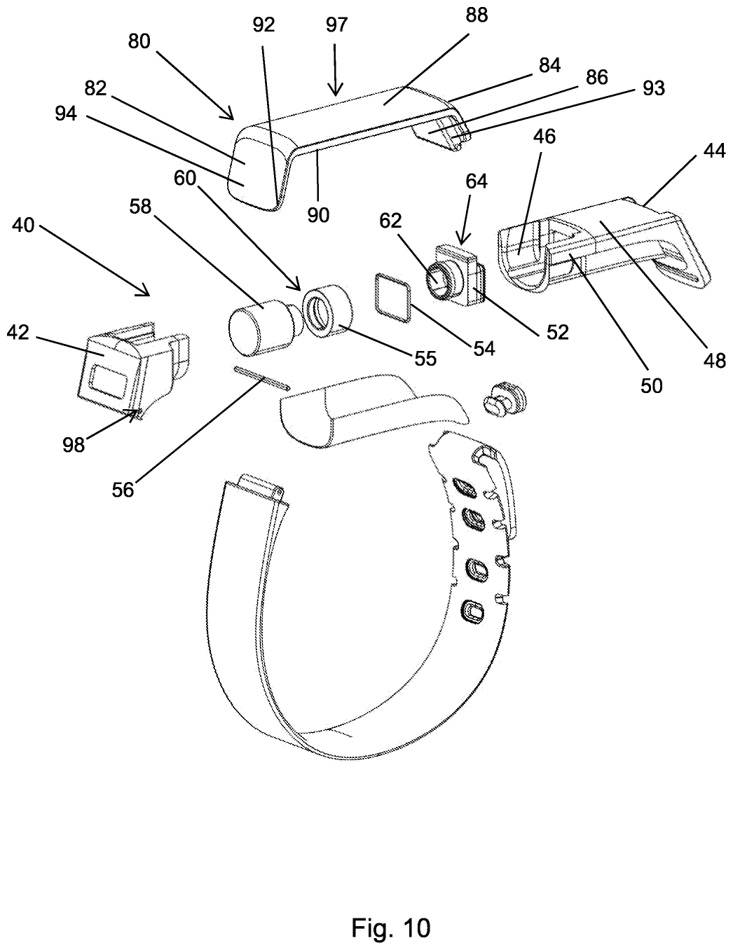

FIG. 11 is a top, perspective view showing the hand sanitizing system, bracelet applicator apparatus, dispensing system, and refill system; the view showing a band; the view showing a sprayer; the view showing an actuator.

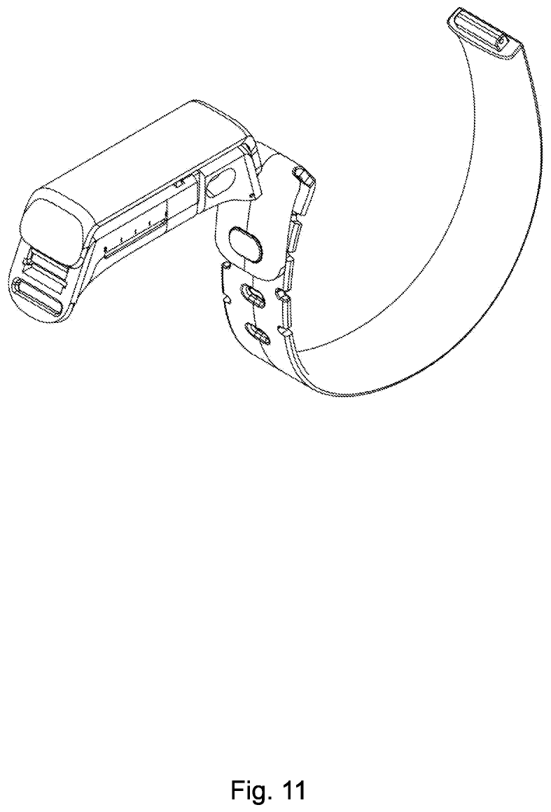

FIG. 12 is a top, perspective view showing the hand sanitizing system, bracelet applicator apparatus, dispensing system, and refill system; the view showing a band; the view showing a sprayer; the view showing an actuator.

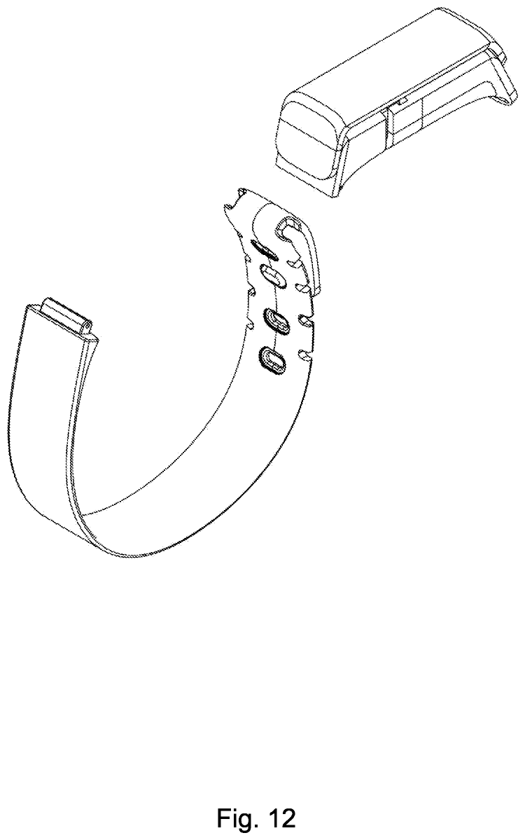

FIG. 13 is a top view showing the hand sanitizing system, bracelet applicator apparatus, dispensing system, and refill system; the view showing a band; the view showing a sprayer; the view showing an actuator.

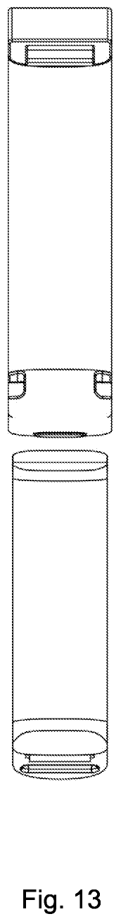

FIG. 14 is a bottom view showing the hand sanitizing system, bracelet applicator apparatus, dispensing system, and refill system; the view showing a band; the view showing a sprayer; the view showing an actuator.

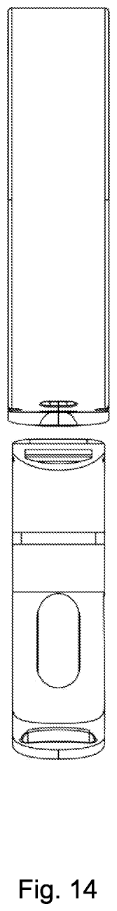

FIG. 15 is a front view showing the hand sanitizing system, bracelet applicator apparatus, dispensing system, and refill system; the view showing a band; the view showing a sprayer; the view showing an actuator.

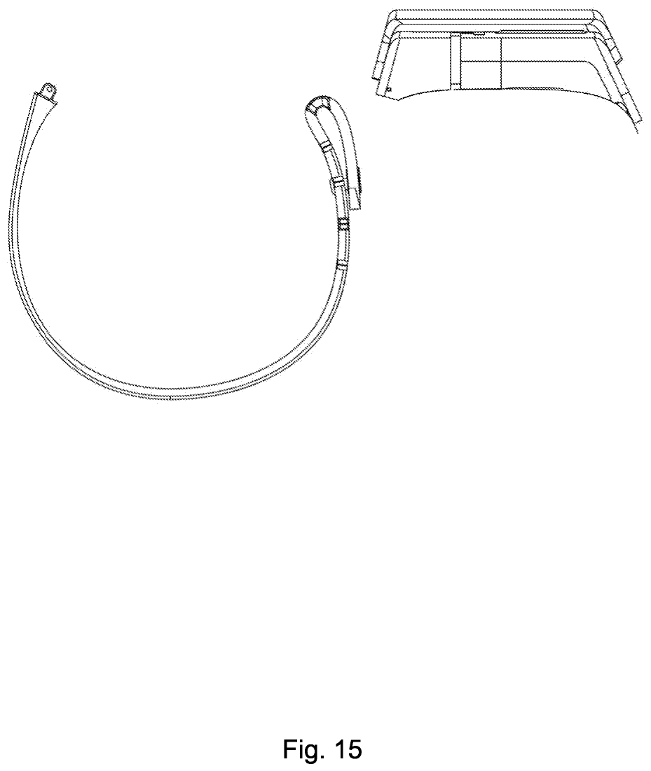

FIG. 16 is a rear view showing the hand sanitizing system, bracelet applicator apparatus, dispensing system, and refill system; the view showing a band; the view showing a sprayer; the view showing an actuator.

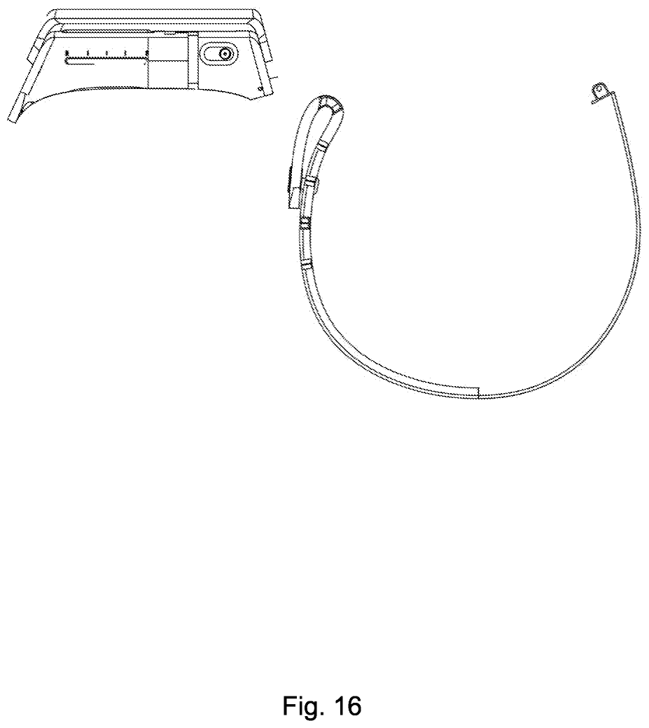

FIG. 17 is a side elevation view showing the hand sanitizing system, bracelet applicator apparatus, dispensing system, and refill system; the view showing a band; the view showing a sprayer; the view showing an actuator.

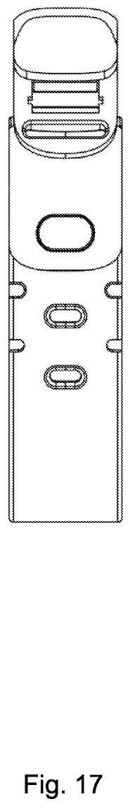

FIG. 18 is a side elevation view showing the hand sanitizing system, bracelet applicator apparatus, dispensing system, and refill system; the view showing a band; the view showing a sprayer; the view showing an actuator.

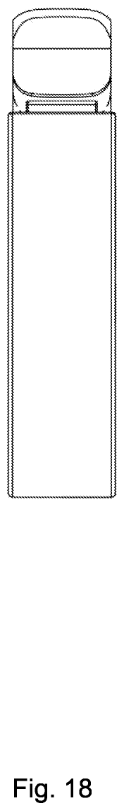

FIG. 19 is a bottom, perspective view showing the hand sanitizing system, bracelet applicator apparatus, dispensing system, and refill system; the view showing a band; the view showing a sprayer; the view showing an actuator.

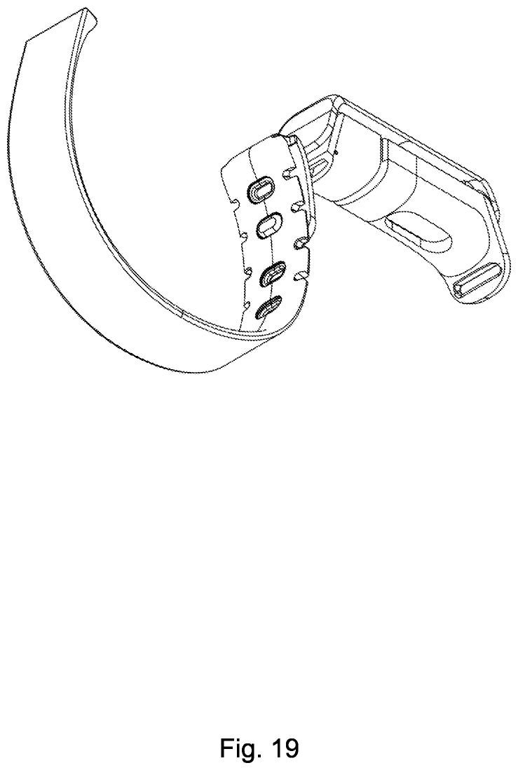

FIG. 20 is a top, perspective view showing the hand sanitizing system, bracelet applicator apparatus, dispensing system, and refill system; the view showing a band; the view showing a sprayer; the view showing an actuator; the view showing a refillable tank.

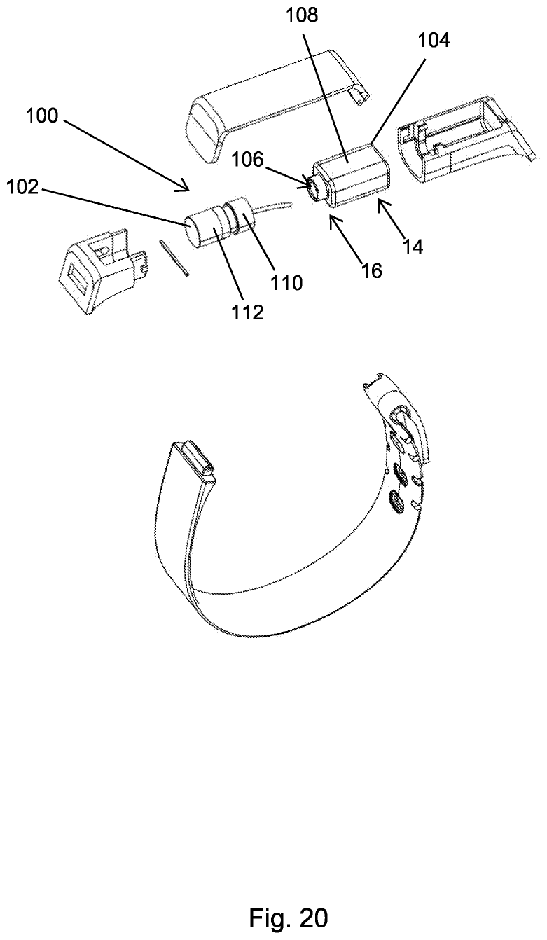

### DETAILED DESCRIPTION OF THE DISCLOSURE

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the disclosure(s). The following detailed description is, therefore, not to be taken in a limiting sense, and the scope of the disclosure(s) is defined only by the appended claims, along with the full scope of equivalents to which such claims are entitled.

As used herein, the terminology such as vertical, horizontal, top, bottom, front, back, end, sides and the like are referenced according to the views, pieces and figures presented. It should be understood, however, that the terms are used only for purposes of description, and are not intended to be used as limitations. Accordingly, orientation of an object or a combination of objects may change without departing from the scope of the disclosure.

Reference throughout this specification to “one embodiment,” “an embodiment,” “one example,” or “an example” means that a particular feature, structure, or characteristic described in connection with the embodiment or example is included in at least one embodiment of the present disclosure. Thus, the appearance of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

All illustrations of the drawings are for the purpose of describing selected versions of the present disclosure and are not intended to limit the scope of the present disclosure.

Although the disclosure has been explained in relation to its preferred embodiment, it is to be understood that many other possible modifications and variations can be made without departing from the spirit and scope of the disclosure.

System

With reference to the figures, a hand sanitizing system, bracelet applicator apparatus, dispensing system, refill system, and method of use 10 are presented herein (also known as “hand sanitizer system”, “bracelet applicator system”, “bracelet disinfectant system”, “dispensing system”, or simply “system). Hand sanitizer system 10 is formed of any suitable size, shape and design and is configured to provide convenient access to a dosage of hand sanitizer for use in sanitizing hands and/or sanitizing a surface. Said another way, bracelet disinfectant system 10 provides a dispensing system for the easy, and convenient application of a sanitizer solution.

Said another way, bracelet disinfectant system 10 is configured as a lightweight wearable. In the arrangement shown herein, as one example, the bracelet disinfectant system 10 is configured to be worn on a user's wrist. In this way, the bracelet disinfectant system 10 is located close to the palm of the user. In this way, the bracelet disinfectant system 10 can eject a predetermined amount of sanitizing solution onto the palm of the user. Furthermore, and in the arrangement shown, as one example, the ejection of the solution can be completed without removing the bracelet from the wrist of the user. Said another way, bracelet disinfectant system 10 can be caused to eject a sanitizing solution from the system 10 to the palm of a user. In this way, a user can quickly and easily eject a predetermined amount of solution onto a single palm and still have both hands free to rub the solution into their hands and/or onto another surface—so as to make the surface disinfected.

In the arrangement shown, as one example, bracelet disinfectant system 10 includes a pump like actuator (to be further discussed herein). The pump like actuator acts as a button and works quickly and efficiently so that a user can press the pump like actuator with ease; forcing an amount of solution to be ejected from the bracelet and on to the palm of the hand of a user.

In this way, and in the arrangement shown herein, a user is likely to wear the bracelet disinfectant system 10 with the nozzle (to be further discussed herein) pointing inwards and/or toward the palm. In this way, when the user presses the actuator, the nozzle sprays solution onto the palm of the user. While this arrangement is hereby contemplated for use, other wearing arrangements, such as wearing the bracelet disinfectant system 10 so the spray lands on the back of the hand of a user. The spray and/or solution can then be rubbed into the hands of the user with both hands free.

Said another way, and in the arrangement shown, as one example, bracelet disinfectant system 10 is formed of a band which straps around the wrist of a user causing the system to fit closely like a wrist watch or wearable device would fit. The bracelet disinfectant system 10 may be thin enough to be worn with other wearable devices, and/or in other embodiments may be thick so as to appear as a standalone wearable. Additionally, and in alternative embodiments, bracelet disinfectant system 10 may incorporate other wearable features such as time telling, step counting, other fitness tracking, global positioning satellites, bluetooth and smartphone and or smart device linking.

In the arrangement shown, as one example, system 10, may comprise a user, a solution 14, and a dispenser apparatus 16, and a cartridge 18, among other components, features, and functionality.

User:

In the arrangement shown, as one example, system 10 includes a user. User is typically a user wearing the bracelet disinfectant system 10 around their wrist. In the arrangement shown, as one example, a user is an individual wishing to apply hand sanitizer to the palm and/or back of the hand, so that the hand sanitizer can subsequently be rubbed into the palm and hand. As another example, a user may be an individual wishing to clean and/or sanitize a surface. In this way, a user wishing to clean and/or sanitize a surface may spray and/or eject the solution onto the surface before clearing the surface. In this arrangement, the user would spray and/or eject the solution past the hand and onto a nearby and/or adjacent surface—such that the surface can be sanitized and/or cleaned.

Solution:

In the arrangement shown, as one example, system 10 includes a solution 14. Solution 14 is formed of any suitable solution and/or any suitable mixture or formula. In the arrangement shown, as one example, and without limitation, solution 14 is a sanitizer. More specifically, and without limitation, the solution shown in one arrangement is a hand sanitizer.

Furthermore, in the arrangement shown, as one example, solution is formed in a spray form of a hand sanitizer. Spray form is one common form of a hand sanitizer. In this way, the solution has the viscosity such that the solution can be dispelled with force to form a spray which emits from the nozzle of the dispenser apparatus 18 (to be further discussed herein). Although a spray is contemplated for use, other embodiments included herein are a liquid and/or liquid spray, a gel, a foam, any combination thereof and the like.

In the arrangement shown, as one example, solution 14 is configured to kill certain types of germs, such as norovirus. Additionally, the solution 14 may be formed to remove chemicals and cleanse, in addition to killing germs and infectious diseases. In one example, the solution 14 is alcohol based, as alcohol based sanitizers are known to be better effective at killing bacteria and the like. Solution 14, as one example, and as an alcohol based sanitizer, may also serve as an antiseptic.

In addition, solution 14 may also contain other properties, such as fragrances—for smell—, dyes—for coloring and aesthetic purposes—, vitamins and minerals—for hand healing properties—, and the like. In this way, the solution can form more purposes other than sanitizing, such as is shown in the various embodiments herein.

In the arrangement shown, as one example, an optimal dosage and/or ejection amount of solution 14 is desired. One quantity hereby considered is 0.3 ml per ejection. However, other quantities may be desired for various applications, such as applications that require additional cleaning and the like (even low doses, such as 0.3 ml, kill 99.9% of bacteria on hands within 30 seconds of the application, and even more within 1 minute). In this way, the optimal dosage may vary from application to application. Additionally, the dosage size may also be changed depending on the cartridge size (to be further discussed herein).

Cartridge:

In one embodiment disclosed herein, system 10 includes a cartridge 16 (cartridge to be further discussed herein).

Dispenser Apparatus:

In the arrangement shown, as one example, system 10 includes a dispenser apparatus 18 (also known as “dispenser system”, or “dispensing system”). Dispenser apparatus 18 is formed of any suitable size, shape, and design and is configured to dispense and/or eject an amount of solution 14. Said another way, the dispenser apparatus 18 is configured to be activated by a user so that the dispenser apparatus 18 can easily eject a solution 14 at a desired time onto a desired surface.

In the arrangement shown, as one example, dispenser apparatus 18 is in the form of a bracelet and/or wearable device. In this way, the dispenser apparatus 18 can be ideally positioned for the application of solution 14 to the surface of the hands of a user. In this way, the dispenser apparatus 18 provides convenient, readily accessible access to the solution 14 and/or hand sanitizer 14 whenever a user needs the solution. In this way, the solution 14 can be readily available on the go and can be used repeatedly.

Additionally, and in the arrangement shown, as one example, dispenser apparatus 18 is refillable. While the dispenser apparatus 18 currently provides for a plurality of doses, the dispenser apparatus 18 can also be refilled. In one embodiment, and as shown herein in one example, the dispenser apparatus 18 includes a cap (cap and/or lid to be further discussed herein) which can be opened and refilled with a hand sanitizer 14 and/or solution 14 of a user's choice. Additionally, and in an alternative embodiment, the dispenser apparatus 18 utilizes replaceable cartridges that come pre-loaded with hand sanitizer 14 and/or solution 14. In this way, if a user runs out of solution 14, held within the cartridge (to be further discussed herein), the user can quickly, and easily pop out the empty cartridge and replace it with a cartridge full and/or partially full of a solution 14. In this way, the dispenser apparatus 18 provides a novel solution delivery system which provides convenience and ease of use.

In the arrangement shown, as one example, the dispenser apparatus 18 is formed of a metal. However, any other material for forming a useful dispenser apparatus and dispenser are hereby contemplated for use. Other materials may include polymers, enhanced polymers, vinyl, polyvinyl chloride (pvc), metals such as aluminum, steel and the like, metal composites, carbon fiber, as well as other combinations of materials that are resistant to destruction while preserving the contents of the interior and maintaining sterility within.

Additionally, and in the arrangement shown, as one example, the dispenser apparatus may be coated in a finish material such as rose gold, gold leafing, gold, platinum, silver, wood, leather, and the like, and other finishing materials.

Band (of Dispenser Apparatus):

In the arrangement shown, as one example, dispenser apparatus 18 includes a band 20 (also known as “wearable band”, “strap”, “securement device”, and “bracelet band”). Bracelet band 20 is formed of any suitable size, shape, and design and is configured to secure the dispensing system 18 to a desired location. In the arrangement shown, as one example, the wearable band 20 is configured to secure the dispensing system 18 to the wrist of a user much like the strap and/or band of a watch would secure a wrist watch to the wrist of a user. In this way, the dispensing system 18 can be secured by the strap 20 to a convenient location on the user.

In this way, and in the arrangement shown, as one example, the dispensing system 18 can be secured adjacent to a wearable device, such as a smartwatch, fitness tracker and the like. Additionally, and in the arrangement shown, as one example, the band 20 can provide securement as a standalone device or with other decorative elements.

In the arrangement shown, as one example, the band 20 extends a length from a first end 22 to a second end 24—where the band secures at each end to the rest of the dispenser apparatus 18. In this way, the attachments located at the first end and the second end are removable and adjustable to fit a variety of wrist sizes and to be interchangeable with other designs and styles for the band. Additionally, and in the arrangement shown, as one example, the band 20 includes an interior surface 26. The interior surface 26 is sized, shaped, and designed to be comfortable for the user. The interior surface 26 may be lined with a different material than the exterior surface 28 so that the interior surface 26 may be softer and/or more comfortable for a user wearing the device. Additionally, and in the arrangement shown, as one example, the band extends between two opposing sides 30. The opposing sides 30 are minimal in thickness so that the band 20 does not protrude an undesirable distance above the surface of the wrist of a user and/or surface of the object the band 20 is secured to.

Additionally, and in the arrangement shown, as one example, the band 20 includes an attachment device 31 (or “attachment feature”). Attachment feature 31 is formed of any suitable size, shape, and design and is configured to provide a plurality of size options and or attachment so the dispenser apparatus 18 can be secured to the wrist and/or removed from the wrist. In this way, the dispenser device 18 can be quickly attached and/or quickly removed from the wrist of a user. Additionally, the attachment feature 31 may provide for a plurality of sizing so that a user can adjust for comfort. In the arrangement shown, as one example, a fastener loop 32 and fastener clip 34 are used to secure and/or remove the dispenser apparatus 18 to the wrist of a user. In the arrangement shown, as one example, the fastener loop 32 and/or fastener clip 34 are familiar shapes to common wristwatch attachments. Additionally, magnets and the slide fasteners and other types of fasteners and/or securement devices are hereby contemplated for use which can attach the dispenser apparatus 18 to an object.

In the arrangement shown, as one example, the band 20 is formed and sized to be aesthetically pleasing. In the arrangement shown, as one example, an aesthetically pleasing, fashionable, high quality leather is used for the band 20 material. In this way, the band 20 appears similar to a stylish and/or luxury end wrist watch. Additionally, and in the arrangement shown, as one example, the band 20 may be formed of a silicone and/or silicone rubber band such that the band can be comfortable and durable and waterproof. While leather bands, silicone bands, silicone rubber bands, and the like are hereby contemplated for use, other materials that are desirable as a band are hereby contemplated for use. Other examples include, but are not limited to, stainless steel straps, nylon banding, polyurethane rubber bands, ceramic band, vegan approved banding, wood bands, titanium, zekapu silicone, among other environmentally friendly bands, a combination thereof, and the like.

Furthermore, in the arrangement shown, as one example, band 20 is formed of a generally slender band. However, band 20 may also be formed of other materials and designs such as a bracelet made of charms, cuffs, jewelry, straps, chains, links, wire, a plurality of rows, pearls, beads, a combination thereof, and the like.

Sprayer (of Dispenser Apparatus):

In the arrangement shown, as one example, dispenser apparatus 18 includes a sprayer 40 (also known as a “dispenser”, a “dispenser portion”, or simply “ejector”). Sprayer 40 is formed of any suitable size, shape, and design and is configured to provide hand sanitizer 14 and/or solution 14, provide the sprayer system which applies the solution, provide the functional button and/or actuator (to be further discussed herein), and more such that the solution 14 can be applied. Said another way, the sprayer 40 consists of many functional components necessary for the application of the solution 14. Said another way, the sprayer 40 refers to the entire system which the band 20 is attached to.

In the arrangement shown, as one example, sprayer 40 extends a length from a first end 42 to a second end 44 and is generally rectangular and box-like in shape with subtle curves and the like for comfort and aesthetic appeal. However, any shape as is necessary for aesthetic and functional purposes is hereby contemplated for use. In the arrangement shown as one example, the sprayer 40 extends from an interior 46, which lines the wrist of a user and/or other object to an outer and/or exterior surface 48 where the actuator is located. Additionally, and in the arrangement shown as one example, the sprayer 40 has opposing sides 50. In the arrangement shown, as one example, the solution 14 is ejected from at least one of the opposing sides 50 such that the solution lands on the palm of the user.

In the refillable embodiment disclosed herein, in the arrangement shown, as one example, the first end 42 includes a cap 52 (also known as “lid”). Cap 52 is formed of any suitable size, shape, and design and is configured to provide access to the refillable tank 58 (to be further discussed herein). The lid 52 is designed to be opened and closed repeatedly and/or when the tank 58 is empty and needs refilled. The lid 52 is designed to fit with close and tight tolerances within the opening of the tank 58 such that no leaks occur and the lid 52 doesn't become open at undesirable times.

In the arrangement shown, as one example, the lid 52 is secured and operably connected by the cap attachment feature 54. Cap attachment feature 54 is configured to allow the cap to open and close while remaining connected to the sprayer 40 so that cap 52 does not become lost and/or disconnected.

In the arrangement shown, as one example, the cap 52 includes a cap release 55. Cap release 55 is formed of any suitable size, shape, and design and may be configured to be a release button and/or a press disengagement feature, which disengages the interlocking engagement of the lid 52 from the sprayer 40. In the arrangement shown, as one example, the cap release 55 is a frictional fit interaction between the opposing sides 50 and at the first end 42 of the sprayer 40.

In this way, the cap release 55 allows the cap 52 to frictionally disengage being locked in place and swing open about a soft connection or hinged connection. Additionally, although a frictional fit is used in the arrangement herein, other forms of engagement are hereby contemplated for use, such as clipping, interlocking mechanisms, hinged connection, and more. Furthermore, as a soft connection, or section of weakened material is used so that the lid 52 can rotate about an axis and open, other connections are hereby considered for use. While connectors are used in the example shown, other connections may be a hinged connection, a spring connection, a joint connection, or other connections which allow for the lid to open.

Additionally, and in the arrangement shown, as one example, the sprayer 40 includes actuator attachment features 56. Actuator attachment features 56 are formed of any suitable size, shape, and design and are configured to secure the actuator 80 (to be further discussed herein) to the sprayer 40. In the arrangement shown, as one example, the actuator 80 is operably attached to the sprayer 40 such that the actuator 80 is configured to interlock and slide across the first end 42 and second end 44 of the sprayer 40. In this way, the actuator 80 operably engages the sprayer 40 while simultaneously activating the dispensing of solution 14. Said another way, when the actuator is pressed and/or forced down vertically, it causes the first end 42 and the second end 44 to slide and/or move in a direction toward one another, decreasing the distance between the first end 42 and the second end 44. In this way, the actuator 80 causes the top of the cartridge 100 (to be further discussed herein) to be pressed, dispensing solution, and/or in the refillable tank embodiment—disclosed herein in one example—to squeeze solution from the tank within and through the nozzle 60, so as to be physically forced from the interior and onto a surface outside of the interior of the sprayer.

Tank: In the arrangement shown, as one example, the sprayer 40 of the dispensing apparatus 18 may also include a plurality of chambers 58 (also known as a “tank” or “refillable tank”). Plurality of chambers 58 is formed of any suitable size, shape, and design and is configured to accept, store, and dispense a solution 14. In the arrangement shown, as one example, a refillable tank 58 includes a top, a bottom, an interior surface, and at least one dispelling aperture and/or opening, among other components and features.

In the arrangement shown, as one example, a single tank 58 is configured in the hollow interior of the sprayer 40 of the dispensing apparatus 18. This refillable tank 58 is generally an elongated chamber extending almost the entire length of the hollow interior of the sprayer 40. In this way, the tank 58 is accessible from the outside through the lid 52 and can be influenced by the applicator 80 (to be further discussed herein). Said another way, the refillable tank 58 is accessible at the portion of the chamber 58 located nearest the lid 52 (to be further discussed herein).

Hollow Tube: In the arrangement shown, as one example, extending a length, and extending from the aperture or opening of the chamber 58 may be a portion forming a hollow tube 62. Hollow tube 62 is formed of any suitable size, shape, and design and is configured as a conveyance for the solution 14 from the interior of the chamber 58 to the spray nozzle 68. In this way, the hollow tube 62 may form as the storage for a single dose amount to be dispelled through the sprayer nozzle 68. In an alternative embodiment, the sprayer 40 may not include a hollow tube 62 and the refillable tank 58 and/or cartridge may be directly attached to the spray nozzle 62.

Additionally, and in the example shown, the sprayer 40 includes a collapsible connection between the first end 42 and the second end 44 such that this portion of the sprayer 40 can be compressed. Said another way, compressing this portion can cause the solution to be dispensed from within the tank and/or cartridge within the sprayer 40. In the arrangement shown, as one example, the solution 14 of the sprayer 40 is dispensed through the sprayer 66 having a sprayer nozzle 68. The solution 14 is dispelled and/or forced through the sprayer opening 70 before landing on a surface.

While this method of dispensing is hereby contemplated for use, other methods of dispensing are also considered. For example, other methods may include a push button which activates a solenoid powered by a power source such as a plurality of batteries. Additionally, yet other arrangements are considered such as a diaphragm which, when pressed, forces solution emission. Furthermore, other dispensations such as the press of a mechanism forcing air into a container as a displacement force for the solution is also hereby contemplated for use. These and other dispensing mechanisms and means are hereby considered.

Actuator (of Dispenser Apparatus):

In the arrangement the dispensing apparatus 18 includes an actuator 80 (or “push button”). Actuator 80 is formed of any suitable size, shape, and design and is configured to control the dispensing action for system 10. Said another way, the actuator 80 is a primary movement component of the dispensing system 18 and is the main interaction of the system 10 with a user by which the user activates the dispensing action. In the arrangement shown, as one example, actuator 80 is formed of a slender, elongated bar like structure which is generally flat on top with an angled first end and second end. In this way, the actuator 80 is designed to be pressed downward onto the sprayer 40 so that the sprayer 40 is compressed. In such a way, the actuator 80 causes the sprayer 40 to decrease in length as measured from the first end 42 of the sprayer 40 to the second end 44 of the sprayer 40. In this way, the solution 14 is caused to dispense. The actuator 80 being configured to cause the solution to dispel.

In the arrangement shown, as one example, the actuator 80 extends a length from a first end 82 to a second end 84, having an interior 86 nearer the sprayer 40 and an exterior surface 88—the exterior surface 88 being considered the top or outermost portion of the dispensing apparatus 18. The actuator 80 also includes opposing sides 90 that generally extend the length of the actuator 80. Additionally, and in the arrangement shown, as one example, the actuator 80 includes an attachment feature 92—plurality of attachment features operably secure the actuator 80 to the sprayer 40. Attachment features may also act as a plurality of guides 93.

In the arrangement shown, as one example, the actuator 80 also includes a closing clip 94 (or “attachment means”, or “locking mechanism”). Closing clip 94 secures the actuator 90 in place when the actuator 80 is not in the open position. Actuator 80 is secured to the sprayer—operably connected—on a first end and can rotate and/or open such that the contents of the interior of the sprayer 40 can be accessed and the actuator 80 can act as a lid to the dispensing system 18—opening and closing as needed. In this way, the actuator 80 may include a closing clip 94, a push release 97, and also a reload spring and/or reload mechanism 98.

In the arrangement shown, as one example, the push release 98 allows the actuator 80 to frictionally disengage being locked in place and swing open about a soft connection or hinged connection. Additionally, although a frictional fit is used in the arrangement herein, other forms of engagement are hereby contemplated for use, such as clipping, interlocking mechanisms, hinged connection, and more. Furthermore, as a soft connection, or section of weakened material is used so that the actuator 80 can rotate about an axis and open, other connections are hereby considered for use. While connectors are used in the example shown, other connections may be a hinged connection, a spring connection, a joint connection, or other connections which allow for the actuator 80 to open and/or close.

While a press and/or mechanical actuator is considered in the arrangement shown, as one example, other actuator types are hereby contemplated for use. Examples of other types of actuators include, but are not limited to, a hydraulic actuator, a pneumatic actuator, an electric actuator, a coiled actuator, a thermal actuator, a magnetic actuator, and a soft actuator, and or an actuator having a comb drive, an actuator having a digital micromirror device, a software controlled actuator in communication with another device via a transceiver, a combination thereof and the like.

Cartridge:

In the arrangement shown, as one example, and in an alternative embodiment, system 10 may include a cartridge 100 (also known as “reloadable cartridge”, or “reloadable capsule”, or simply “capsule”). Reloadable cartridge 100 is formed of any suitable size, shape, and design and is configured to house and hold a solution 14. Additionally, cartridge 100 is configured to fit, with close and tight tolerances to prevent shifting, within the interior of the sprayer 40. Additionally, and in the arrangement shown, as one example, the cartridge is designed and configured to be removed from the interior of the sprayer 40 and replaced by a loaded cartridge. This process and cartridge 100 is configured to repeat such that a full cartridge and/or partially full cartridge 100 can replace an empty and/or used cartridge.

In the arrangement shown, as one example, cartridge 100 is formed such that it looks like a small spray bottle having a container and/or housing for the solution 14 and a lid and/or cap 110 which also includes a sprayer 112 and/or nozzle 114. The cartridge 100 extends a length from a first end 102 to a second end 104, having a hollow interior 106 for housing the solution 14, and an exterior surface designed to fit without slippage within the interior of the sprayer 40 and or slide in and out of the interior of the sprayer 40.

In the arrangement shown, as one example, cartridge 100 is formed of a generally elongated square as from the bottom up or the top down. The cartridge 100 includes softened edges for ease of handling and ease of replacing cartridges and/or moving and/or removing cartridges from the hollow interior of the sprayer 40.

### In Operation/Methods of Use

### System Embodiments

Refill System:

In the arrangement shown, as one example, as is shown in another embodiment, the refill mechanism and/or the cap and/or lid is formed from the top of the sprayer. The chamber of the hollow interior is refilled with a solution by opening the actuator, removing the sprayer and exposing the chamber. The chamber can then be refilled with solution and/or filled with solution before reattaching the sprayer and closing the actuator.

Refillable System:

As one example, system 10 and methods of use include a refillable tank embodiment. This embodiment utilizes an actuator for dispensing and applying a solution to hands and/or another surface. This embodiment includes a refillable tank with a cap. The cap can be opened and the refillable tank can be filled with a solution. This process can be repeated as needed.

Cartridge Replacement System:

As another example, system 10 and methods of use include a cartridge replacement embodiment. This embodiment utilizes an actuator for dispensing and applying a solution to hands and/or another surface. This embodiment includes a replaceable cartridge. The dispensing system 18 can be opened, a cartridge with solution placed inside. The cartridge with solution can be used. When empty or low, the empty or low cartridge can be replaced with another cartridge which is full and/or contains solution.

These and other embodiments and methods of use are hereby contemplated for use.

It will be appreciated by those skilled in the art that other various modifications could be made to the device without parting from the spirit and scope of this disclosure. All such modifications and changes fall within the scope of the claims and are intended to be covered thereby.

What is claimed:

1. A hand cleansing system, the system comprising:
a dispenser apparatus;
a band;
the band extending a length from a first end to a second between opposing sides;
the band having an interior surface and an exterior surface;
a sprayer;
the sprayer extending a length from a first end to a second end;
the sprayer having a cap;
the sprayer having a tank;
the sprayer having a nozzle;
the sprayer having an opening;
wherein the tank provides storage for the solution;
wherein the tank is operably connected to the nozzle;
wherein the nozzle is configured to point and spray through the opening of the sprayer;
an actuator;
the actuator extending a length from a first end to a second end;
the actuator configured to compress the tank of the sprayer when the actuator is pressed;
the actuator operably connected such that pressing the actuator causes the tank to be compressed by pressure;
wherein pressure on the actuator, operably connected, causes the solution to dispense from the nozzle of the sprayer;
wherein pressure on the actuator, operably connected, causes the solution to dispense through the opening of the sprayer;
wherein the hand cleansing system is refillable.

2. The system of claim 1, further comprising:
a solution.

3. The system of claim 1, further comprising:
a solution;
wherein the solution is a hand sanitizing solution.

4. The system of claim 1, further comprising:
the band having an attachment device;
wherein the band attaches to the dispenser apparatus at the first end of the band and the second end of the band.

5. The system of claim 1, further comprising:
wherein the band is configured to secure the dispenser apparatus to a wrist of a user.

6. The system of claim 1, further comprising:
the band having a fastener loop.

7. The system of claim 1, further comprising:
the band having a fastener clip.

8. The system of claim 1, further comprising:
the sprayer having a cap attachment feature;
wherein the cap attachment feature operably connects the cap to the tank.

9. The system of claim 1, further comprising:
the sprayer having a cap release feature;
wherein the cap release feature is configured to release the cap from the tank.

10. The system of claim 1, further comprising:
the actuator having a plurality of guides;
wherein the plurality of guides are operably configured to provide smooth operation of the actuator as the actuator slides into an engagement position and out of an engagement position.

11. The system of claim 1, further comprising:
the actuator having a plurality of guides;
wherein the plurality of guides are operably configured to provide smooth operation of the actuator as the actuator slides into an engagement position and out of an engagement position;
the actuator operably connected to a spring;
wherein the spring causes the actuator to move to a disengaged position autonomously after the actuator has been pressed into the engagement position.

12. The system of claim 1, further comprising:
a push release;
wherein the push release is operably connected to the actuator;
wherein pressing the push release causes the actuator to open;
wherein the interior components of the dispenser apparatus can be accessed when the actuator is open.

13. A bracelet applicator apparatus, comprising:
a cartridge;
the cartridge extending a length from a first end to a second end;
the cartridge having a tank;
wherein the tank provides storage for the solution;
a dispenser apparatus;
a band;
the band extending a length from a first end to a second between opposing sides;
the band having an interior surface and an exterior surface;
the band having an attachment device;
wherein the band attaches to the dispenser apparatus at the first end of the band and the second end of the band;
an actuator;
the actuator extending a length from a first end to a second end;
the actuator configured to compress the cap of the cartridge when the actuator is pressed;
wherein the cartridge of the bracelet applicator apparatus is replaceable.

14. The apparatus of claim 13, further comprising:
a solution.

15. The apparatus of claim 13, further comprising:
a solution;
wherein the solution is a hand sanitizing solution.

16. The apparatus of claim 13, further comprising:
the band having an attachment device;
wherein the band attaches to the dispenser apparatus at the first end of the band and the second end of the band.

17. The apparatus of claim 13, further comprising:
the cartridge having a sprayer;
the sprayer extending a length from a first end to a second end;
the sprayer having a cap;
the sprayer having a nozzle;
the sprayer having an opening;
wherein the tank is operably connected to the nozzle;
wherein the nozzle is configured to point and spray through the opening of the sprayer.

18. The apparatus of claim 13, further comprising:
the actuator operably connected such that pressing the actuator causes the cap of the cartridge to compress a hollow interior of the tank;
wherein pressure on the actuator, operably connected, causes the solution to dispense from the nozzle of the sprayer;
wherein pressure on the actuator, operably connected, causes the solution to dispense through the opening of the sprayer.

19. The apparatus of claim 13, further comprising:
the actuator having a plurality of guides;
wherein the plurality of guides are operably configured to provide smooth operation of the actuator as the actuator slides into an engagement position and out of an engagement position;
the actuator operably connected to a spring;
wherein the spring causes the actuator to move to a disengaged position autonomously after the actuator has been pressed into the engagement position.

20. A method of cleansing the hands of a user, comprising the steps:
providing a dispenser apparatus; the dispenser apparatus having a band attached thereto; the dispenser apparatus having a sprayer; the dispenser apparatus having a solution; the dispenser having an actuator;
positioning the dispenser apparatus such that the sprayer will cause a solution to land on the surface of the hands of a user;
pressing the actuator;
dispensing solution;
rubbing solution into the hands of the user.
